(19) United States
(12) Patent Application Publication
(10) Pub. No.: US 2016/0183800 A1
(43) Pub. Date: Jun. 30, 2016

(54) DEVICE AND METHOD FOR MEASURING THE ELASTICITY OF A MACROSCOPIC SAMPLE

(71) Applicant: EBERHARD KARLS UNIVERSITÄT TÜBINGEN, Tübingen (DE)

(72) Inventors: Tilman Schäffer, Tübingen (DE); Andreas Kirschniak, Tübingen (DE)

(73) Assignee: Eberhard Karls Universitat Tubingen, Tubingen (DE)

(21) Appl. No.: 14/910,811

(22) PCT Filed: Aug. 7, 2014
(86) PCT No.: PCT/EP2014/067000

§ 371 Date: Feb. 8, 2016

(30) Foreign Application Priority Data
Aug. 7, 2013 (DE) 10 2013 108 512.1

(51) Int. Cl. A61B 5/00; A61B 17/00; A61B 17/50; A61B 34/30; A61B 17/3203

(52) U.S. Cl. CPC A61B 5/0053; A61B 34/30; A61B 17/3203; A61B 17/50; A61B 17/00234; A61B 2017/00057; A61B 2217/005; A61B 2217/007; A61B 2017/0003

(57) ABSTRACT

A device for measuring the elasticity of a macroscopic sample, in particular for measuring the elasticity of tissue of a living human or animal, is disclosed, which comprises the following: at least one outlet for a jet of fluid and/or one inlet for aspiration of a stream of fluid, means for positioning the device in relation to the macroscopic sample in such a way that the outlet and/or the inlet and/or an end face of the device is at a predetermined distance from the macroscopic sample, or at a distance therefrom that can be determined by said means, and a mechanism for measuring a variable that is characteristic of the extent of a deformation of the sample on account of an interaction of the sample with the jet of fluid and/or the aspirated stream of fluid, wherein this variable is determined by an ion current in the deformation area of the sample, or the volumetric flow of the fluid itself, or, if the fluid is enclosed by the elastic sample, by a change of volume and associated change of pressure in the enclosed fluid.

### FIELD OF THE INVENTION

The present invention relates to a device and a method for measuring the elasticity of a macroscopic sample, which in particular may be the tissue of a living human or animal. In particular, the present invention relates to a surgical or diagnostic instrument which equips a device of this type.

### BACKGROUND OF THE INVENTION

In the event of surgical interventions it is often the responsibility of the treating surgeon to distinguish between what is benign tissue and what is malignant tissue. In order to assist the surgeon in making this decision, what is known as the frozen section analysis is often currently performed, this being a pathological analysis of tissue samples during an ongoing operation. Frozen sections are produced from the removed tissue sample and are promptly dyed and examined by a pathologist. The frozen section analysis is currently the gold standard for intraoperative assessment of removed tissue. The main disadvantage of the frozen section analysis, however, is that the operation is considerably lengthened, even with optimal working practices. A further disadvantage lies in the fact that the morphological quality of frozen sections is poorer than that of conventional histological sections, in which case the tissue is fixed in paraffin or another synthetic material.

In order to avoid the need for frozen section analysis there is a great interest in the development of dye-free analysis techniques which can be applied directly in the operating theatre. In particular, methods for measuring elastic tissue properties have recently been applied increasingly in tissue differentiation. With ultrasound elastography (Sarvazyan A, G. D., Maevsky E, Oranskaja G, Elasticity imaging as a new modality of medical imaging for cancer detection: Proceedings of International Workshop on Interaction of Ultrasound with Biological Meda, 1994; p, 3) and (Venkatesh, S. K. M Jin, J. F. Glockner, N Takahashi, P. A. Araoz, Talwalkar, et al, mit Magnetresonanz-Elastographie MR elastography of liver tumors: preliminary results. American journal of roentgenology, 2008. 190(6): p. 1534-40) it has already been demonstrated that the rigidity of the tissue, i.e. the E-modulus thereof, can be used for the identification of tumours. A multiplicity of devices and methods have subsequently been proposed, with which the elasticity of human tissue can be measured in vivo, for example using a torsional resonator device (Valtorta D, E. Mazza, 2006, Measurement of Rheological Properties of Soft Biological Tissue with a Novel Torsional Resonator Device, Rheological Acta, 45(5), 677-692) or by aspiration by means of negative pressure (Gran P., 2007, Aspiration Experiment: Analysis of in Vivo Measurements on Human Liver and Comparison with the Indentation Experiment. Report Number ETH/ZfM-2008/03, February 2008, ETH Zurich). Besides mechanical approaches of this type, optical devices and methods have also been proposed, for example with use of confocal microscopy (Snedeker J G, Ben-Arav A, Zilberman Y, Pelled G, Gazit D (2009) Functional Fibered Confocal Microscopy: a Promising Tool for Assessing Tendon Regeneration. Tissue Eng Part C15(3): 485:491) or with use of optical coherence tomography. An overview of different techniques is provided in Li, Y, Snedeker, J G., Elastography: modality-specific approaches, clinical applications, and research horizons. Skeletal Radiol (2011) 40:389-397.

Currently, ultrasound-based elasticity measuring devices or “elastography” devices in particular have achieved market maturity.

All known devices and methods for the in vivo elasticity measurement of human or animal tissue have the disadvantage, however, that they are associated with a comparatively high equipment outlay.

### SUMMARY OF THE INVENTION

The object of the invention is to specify a device and a method for measuring the elasticity of a macroscopic sample, in particular for measuring the elasticity of tissue of a living human or animal, which device and method enable a precise elasticity measurement with comparatively low equipment outlay.

The term “elasticity” is to be understood broadly in the present disclosure and includes all aspects of the way in which the sample reacts to pressure, shear forces and the like. In particular, but without limitation, the measurement of the elasticity may include the at least approximate determination of one or more characteristic parameters, such as a modulus of elasticity (E-modulus) or the shear modulus or variables related thereto. However, the elasticity measurement may also include the at least approximate determination of some or all components of the elasticity tensor of the sample. The elasticity measurement may however also include the determination of one or more parameters that form the basis of a model of the tissue properties, inclusive of the viscoelastic properties of the tissue. For the sake of simplicity, all of these aspects of the mechanical properties of the tissue will be referred to hereinafter as “elasticity”, and this term will also be understood in the present disclosure in accordance with this general definition.

Here, the term “macroscopic sample” is intended to convey the idea that the sample is not constituted by individual cells or small cell clusters that can be examined only in an isolated manner in a laboratory. Rather, the macroscopic sample is typically a region of human tissue of a living patient, measuring several cm² in size, possibly even more than 100 cm² in size.

This object is achieved by a diagnostic or surgical instrument according to claim 1 and a method according to claim 20. Preferred exemplary embodiments are defined in the dependent claims.

The diagnostic or surgical instrument comprises a device for measuring the elasticity of the macroscopic sample, in particular for measuring the elasticity of tissue of a living human or animal. Here, the device comprises at least one outlet for a jet of fluid and/or one inlet for aspiration of a stream of fluid. The device also comprises means for positioning the device in relation to the macroscopic sample in such a way that the outlet and/or the inlet or an end face of the device is at a predetermined distance from the macroscopic sample, or is at a distance therefrom that can be determined by said means. Lastly, the device comprises a mechanism for measuring a variable that is characteristic of the extent of a deformation of the sample on account of an interaction of the sample with the jet of fluid and/or the aspirated stream of fluid. Herein, this variable is determined either by an ion current in the deformation area of the sample or the volumetric flow of the fluid itself. If the fluid is enclosed by the elastic sample and the stream of fluid is therefore completely dammed up, the variable characteristic of the deformation can be determined from a change in volume and an associated change in pressure in the enclosed fluid.

In accordance with the invention, the sample is therefore artificially deformed on account of an interaction with the jet of fluid and/or with the aspirated stream of fluid. The term “interaction” of the sample with the jet of fluid/stream of fluid indicates that not only does the sample deform, but the jet of fluid can also be influenced by the interaction, in particular can be slowed down or also completely dammed up, for example when the sample bears so tightly against the device in an annular region around the outlet that no fluid or only insignificant quantities of the fluid can escape therebetween.

The deformation can be produced in different ways, for example by pulse or momentum transfer of an impinging hard jet of fluid, by deformation as a result of a dynamic pressure of a stream in the deformed region, or by a hydrostatic pressure, which is produced when the fluid is enclosed in a bubble-like manner by the elastic sample, the jet of fluid is thus dammed up, and the flow comes to a standstill. The extent of the deformation is a measure here for the elasticity of the sample. In accordance with the invention the extent of the deformation of the sample is measured via a variable that is characteristic of the deformation in one of two possible ways.

On the one hand this variable is determined by an ion current in the deformation area of the sample. Here, the term “is determined” indicates that the specified variable may relate to the ion current itself or to another variable, provided this relates clearly to the ion current in an unambiguous manner and therefore “is determined” thereby. As will become clearer on the basis of the exemplary embodiments described below, a direct or indirect ion current measurement can be combined easily and with low equipment outlay with the artificial deformation by a jet of fluid or a stream of fluid. The ion current in many cases will actually flow through the fluid itself. Both the mechanical deformation and the measurement of the deformation of the same medium are used for this purpose, which further promotes the simplicity of the structure.

In a second embodiment the variable characteristic of the deformation of the sample is determined by the volumetric flow of the fluid itself, which in turn enables an extremely simple structure in terms of the equipment. This is based on the finding that the volumetric flow is likewise dependent on the deformation of the sample because the hydrodynamic resistance of the fluid in the deformed area is dependent on the extent of the deformation. Besides the elasticity of the sample, the volumetric flow is of course also dependent on the pressure of the fluid, and equally the ion current is also dependent on the applied voltage. In practical applications the volumetric flow is therefore determined at a predefined pressure of the fluid or generally depending on the fluid pressure. It is also possible to keep the stream of fluid constant and to determine the corresponding fluid pressure. In all of these cases, however, the volumetric flow is a variable characteristic of the deformation, and all of these variants should be covered by the feature.

In the event that the fluid is enclosed completely or nearly completely by the elastic sample and the volumetric flow thus comes to a standstill, the volumetric flow is of course no longer suitable for measuring the extent of the deformation. In this case, however, a change in volume and an associated change in pressure can be measured in the enclosed fluid, for example. By way of example, an additional quantity of fluid can be “pumped” into the bubble enclosed by the sample, and the associated pressure increase can be measured. With a given additional volume quantity the stiffer is the sample, the more will the pressure rise. Alternatively, the applied pressure can of course also be increased, and it is possible to measure how much more fluid flows into the bubble. However, it should be taken into consideration that the measurement of the deformation can be taken with the aid of the ion current measurement both in cases in which a continuous stream of fluid is produced in the deformation area of the sample and in cases in which the fluid is dammed up and the jet of fluid comes to a standstill.

Both the extent of the deformation and the ion current or the volumetric flow of the fluid are dependent in practice, however, on the distance of the device from the macroscopic sample. In order to provide quantitative measurements, means are therefore provided for positioning the device in relation to the macroscopic sample in such a way that the outlet and/or the inlet and/or an end face of the device is/are at a predetermined distance from the macroscopic sample. Alternatively, it may be sufficient if the distance can be determined by the aforementioned positioning means, whereby the measurement results can then be calibrated in accordance with the actual distance. A special case is constituted by a device that vibrates at least in portions with respect to the sample or is moved to and fro, such that the distance between the outlet and/or the inlet and the sample fluctuates, in combination with an ion current measurement. By means of the ion current measurement, a characteristic distance or distance range can be detected, in which the ion current is “cut off” when the device or the corresponding portion, for example a nozzle, is moved sufficiently closely towards the sample. The stiffer is the sample, the more abrupt does this cut-off effect occur. In such an embodiment the distance can therefore be measured (via the occurrence of the cut-off effect), as can the elasticity (for example via the difference quotient from distance and ion current change).

The device preferably has an end face that can be arranged parallel or approximately parallel to the surface of the macroscopic sample so as to form a gap with the surface of the macroscopic sample. The size of this gap can then be varied by the jet of fluid or the stream of fluid depending on the elasticity of the sample, which in turn can be detected by a variation in the ion current or in the volumetric flow of the fluid. The end face can also be designed to bear against the surface of the macroscopic sample during operation of the device, and in this case the above-mentioned “predetermined distance” between the end face and the sample is zero. Here as well an open gap between the sample and the end face can be formed by the jet of fluid, through which the fluid escapes, or a “closed gap”, specifically a closed fluid bubble, can be formed, such that the jet of fluid is dammed up. In both cases, however, a deformation of the sample is in turn produced and is characteristic of the elasticity of said sample.

The device preferably has a continuous or interrupted bearing surface, by means of which the device can be placed on the sample during the measurement of the elasticity, wherein the bearing surface occupies an area of at least 10 mm², preferably at least 30 mm², and particularly preferably at least 1 cm². An example of an “interrupted” bearing surface would be for example a bearing surface that is formed by a plurality of individual bearing elements or spacers. The area “occupied” by the interrupted bearing surface here designates the surface area of a continuous area in which the interrupted area can be contained. If the interrupted area is formed for example by four bearing elements arranged in a square, which has a side length of 1 cm, the “occupied” area would thus be 1 cm². A comparatively large bearing surface makes it possible to guide the device over the sample with contact and in particular to guide the device in a sliding manner along the sample.

In an advantageous embodiment the fluid is an electrolyte, in particular a saline solution. In this case the specified ion current can flow directly through the fluid. The outlet and/or the inlet is/are preferably arranged in the end face.

In an advantageous development the device comprises at least one first and at least one second electrode, between which a voltage can be applied, and a current measuring mechanism for measuring an electric current flowing between the first and the second electrode. Here, the at least one first electrode and the at least one second electrode are arranged such that, with suitable positioning of the device in relation to the macroscopic sample, at least some of the current can be formed by an ion current in an electrolyte in a gap between the device and the macroscopic sample. Here, this gap may be in particular a gap that is only produced by the deformation of the sample.

The at least one first electrode is preferably arranged in a fluid channel that is connected to the outlet, and/or the at least one second electrode is preferably arranged in a fluid channel that is connected to the inlet. Here, at least one first and/or at least one second electrode can be formed by a conductive coating of at least part of the respective fluid channel. This embodiment has not only manufacturing advantages, but also prevents the electrode from significantly increasing the hydrostatic resistance in the channel.

The device preferably comprises at least two second electrodes, particularly preferably at least three second electrodes, and in particular at least four second electrodes, wherein a voltage can be applied between each of the second electrodes and the at least one first electrode. By comparing the currents through the respective second electrodes, additional information relating to the lateral variation of the deformation and/or relating to the orientation of the device with respect to the sample can be obtained.

The at least one first electrode and/or the at least one second electrode is/are preferably arranged in an indentation in the end face or in a channel that leads into the end face. This arrangement of the electrodes allows a precise measurement, without the electrodes interfering with the positioning of the device in relation to the sample.

In an advantageous development the at least one first electrode is arranged in a radially inner portion of the end face, and the at least two second electrodes are arranged in or in the vicinity of different, radially outer portions of the end face. This special arrangement of the first and the at least two second electrodes opens up a number of advantages both with regard to the measurements of the extent of the deformation and with regard to the positioning of the device in relation to the macroscopic sample. By way of example, the means for positioning the device in relation to the sample can be designed to detect a tilting of the end face in relation to the surface of the macroscopic sample by means of comparison of the currents through the at least two second electrodes. This will be explained in greater detail hereinafter on the basis of an exemplary embodiment. It should be taken into consideration that in this case the ion current measurement is used for the positioning of the device in relation to the sample, i.e. that the ion current measurement can be used within the scope of the invention not only to measure the extent of the deformation.

If the ion current measurement is used to measure the extent of the deformation, the at least one first and the at least one second electrode are preferably arranged in relation to the outlet and/or inlet such that at least some of the electric current between the at least one first and the at least one second electrode can be formed by an ion current in the deformed area of the sample. Here, the parameter characteristic of the deformation is formed by the current flow between the at least one first and the at least one second electrode.

In an advantageous development the means for positioning the device in relation to the macroscopic sample comprise at least one spacer. This spacer then automatically fixes the predetermined distance between the outlet or inlet and the sample. The at least one spacer is preferably arranged annularly around the outlet or inlet. Here, the spacer by way of example may itself have the form of a ring or a broken ring, or a plurality of spacers may be provided, which are arranged along a ring around the outlet or inlet.

The at least one spacer preferably protrudes beyond the end face. A gap of defined size is thus formed between the end face and the sample in a simple manner, wherein the size of the gap is dependent on how far the spacers protrude beyond the end face.

In an alternative embodiment the second electrode is arranged on the end face and the means for positioning the device in relation to the macroscopic sample are formed by the end face itself, which in this case is to be placed against the macroscopic sample. When the end face with the second electrode arranged thereon bears directly against the sample, no ion current or at most a small ion current can flow through the second electrode. If, however, a jet of fluid is directed to the sample, the sample lifts from the end face as a result of deformation and thus lifts from the second electrode, such that parts of the second electrode are exposed and can be reached by an ion current. In this respect the ion current in this embodiment as well is directly dependent on the extent of the deformation of the sample and is thus suitable for determining the extent of the deformation.

In an advantageous development the device has a plurality of outlets or inlets arranged side by side. Here, the number of outlets or inlets may be at least 4, preferably at least 10, particularly preferably at least 50, and in particular at least 100. Here, at least the majority of the outlets or inlets are assigned respective electrodes for ion current measurement. By means of a multiplicity of outlets/inlets arranged side by side, the sample can be examined simultaneously at a multiplicity of points, such that a spatially resolved elasticity distribution can be measured, even if the device is not moved. This is of exceptional practical advantage for the user, because the user with comparatively little movement of the device in relation to the sample can examine comparatively large areas in a spatially resolved manner and in particular can be comparatively sure that certain anomalies in the elasticity do not remain hidden. This is an advantage compared with devices with which the elasticity can be measured only at certain points, because it is then left to the user to select the measurement points, i.e. it is left to a certain extent to the overview and intuition of the user as to whether all relevant positions have actually been examined. With a device that has an array of outlets/inlets arranged side by side, the sample by contrast can be examined over a large area and it can be ensured comparatively easily that all relevant areas have also actually been measured.

In an advantageous development the outlet is formed by a nozzle, in particular by a nozzle of which the cross section, shape and/or exit angle is/are adjustable. Modifications to the nozzle of this type allow differentiated elasticity measurements.

In an advantageous embodiment the device comprises a mechanism for generating a pressure in a channel that is connected to the outlet and/or a mechanism for generating a negative pressure in a channel that is connected to the inlet. Here, the mechanism for generating a pressure may comprise an external pressure source that is connected to the device by a pressure line. This is a simple and comparatively robust solution from a design viewpoint. In an alternative embodiment the pressure generation mechanism is provided in the device itself, such that the pressure can be generated self-sufficiently. Pressure generation mechanisms that comprise one or more piezoelectric actuators in order to generate the pressure, similarly to the manner known for example from inkjet printers, are particularly suitable for this purpose.

In a further preferred embodiment the pressure generation mechanism comprises an exchangeable cartridge that is arranged within the device and that is filled with a pressurised fluid, in particular gas. The generation of the pressure with a cartridge of this type is of simple design similarly to the variant with an external pressure generation mechanism, but has the advantage that a pressure line can be omitted here as well and the device can be handled self-sufficiently and easily. A cartridge of this type can also be provided outside the device and may therefore form an external pressure generation mechanism.

The mechanism for generating the pressure is preferably suitable for generating a time-dependent pressure profile and/or the mechanism for generating the negative pressure is preferably suitable for generating a time-dependent negative pressure profile. The mechanism for measuring the variable characteristic of the deformation of the macroscopic sample is also suitable for measuring this variable in a time-resolved manner. Not only can stationary properties, such as the modulus of elasticity, therefore be determined, but also viscoelastic properties of the sample.

In an advantageous development the device comprises at least two second electrodes and the device comprises a data processing unit that is suitable for determining a lateral variation in the elasticity by comparison of the currents through the at least two second electrodes. Additional information that goes beyond a spatially resolved elasticity measurement at certain points is thus obtainable. In particular, this functionality is useful when determining the profile of the boundaries between tumour and healthy tissue.

When the device does not itself have a data processing unit of this type, it can alternatively be connected to a data processing unit.

As mentioned in the introduction, the extent of the deformation can be determined not only by an ion current, but also by the volumetric flow of the fluid itself. In an advantageous embodiment the variable characteristic of the deformation of the sample is therefore formed by a combination of a volumetric flow of the jet of fluid and the pressure applied in order to generate the jet of fluid, which effectively amounts to the measurement of a hydrodynamic resistance or a variable related thereto. This will also be made clearer hereinafter on the basis of an exemplary embodiment.

As mentioned in the introduction the invention relates to a diagnostic or surgical instrument that is to be guided by hand or by a surgery robot, wherein the instrument comprises a device according to one of the above-described embodiments. A hand-guided instrument of this type allows the doctor to perform the elasticity measurements at precisely the positions at which he suspects tumour tissue or a boundary between tumour tissue and healthy tissue. A hand-guided instrument of this type is optimally suitable for the examination of macroscopic samples because points of interest or suspicious points of the sample can be examined therewith selectively in a spatially resolved manner. In this context, it should be taken into consideration that the reference to macroscopic samples does not necessary imply a low spatial resolution of the individual elasticity measurements. By way of example, it is easily conceivable that highly spatially resolved elasticity measurements are performed with a very fine jet of fluid, and at the same time the hand-guided apparatus nevertheless allows the examination of a comparatively large macroscopic sample, within which the doctor can select the special areas to be examined, for example tissue portions suspected of constituting tumours. Instead of a “hand-guided” instrument, however, an instrument which is guided by a surgery robot may also be used.

In a particularly advantageous embodiment the instrument is also designed for water jet surgery. In the case of water jet surgery, tissue is cut using a fine high-pressure jet of water, this having a series of practical advantages. The combination of the device according to the invention with an apparatus of this type provides immense practical advantages because the components provided anyway for the water feed, pressure generation, nozzles, etc. can be used for the generation of the jet of fluid for the local deformation of the tissue. The additional equipment outlay is extraordinarily low both in respect of the costs and in respect of the installation space.

In an advantageous embodiment the instrument comprises a probe, tweezers, a cutting edge and/or tongs, i.e. instruments that can be used for surgical treatment or for biopsy. By way of example, this makes it possible to identify and directly remove a tumour tissue using the same instrument. These specified tools preferably have an HF terminal for electrosurgical functions, which provide further advantages, for example the cauterisation of cuts and the like.

In an advantageous development the instrument also comprises a camera. Here, the instrument is connected or can be connected to a data processing unit that relates the elasticity values measured in a location-dependent manner to the images recorded by the camera. By way of example, it would be possible to superimpose the recorded image with an “elasticity image” by highlighting in colour areas in which specific changes in the elasticity have been found in order to thus identify tumour tissue more easily.

In a particularly advantageous embodiment the instrument is an endoscopic or laparoscopic instrument. Endoscopes and laparoscopes are also considered in the present disclosure to be hand-guided diagnostic or surgical instruments and constitute particularly advantageous applications for the device according to the invention for elasticity measurement. Here, the device for elasticity measurement is preferably formed by an adjustable, in particular rotatable measuring head of the endoscopic or laparoscopic instrument. In this way, the tried and tested minimally invasive diagnosis, biopsy and ablation of tumour tissue by means of endoscope or laparoscope can be extended by the function of elasticity measurement, which assists the reliable identification of tumour tissue.

### BRIEF DESCRIPTION OF THE DRAWINGS

FIG. 1a shows a schematic sectional view of a device for elasticity measurement in accordance with an embodiment of the invention.

FIG. 1b shows a view from below of the device from FIG. 1a.

FIG. 2 is a schematic illustration illustrating the deformation of tissue by a jet of fluid.

FIG. 3 is a schematic sectional view of a further device for elasticity measurement.

FIG. 4 is a schematic sectional view of a device for elasticity measurement having an annular spacer.

FIG. 5 is a schematic sectional view of a device for elasticity measurement having a spacer formed by a flexible rubber lip.

FIG. 6a is a schematic sectional view of a further device for elasticity measurement in accordance with an embodiment of the invention.

FIG. 6b is a view from below of the device from FIG. 6a.

FIG. 6c shows a schematic sectional view of a further device for elasticity measurement in accordance with an embodiment of the invention.

FIG. 6d shows a schematic sectional view of a further device for elasticity measurement in accordance with an embodiment of the invention.

FIG. 6e shows a detail of a further device for elasticity measurement in accordance with an embodiment of the invention.

FIG. 7 is a schematic sectional view of a further device for elasticity measurement having two second electrodes.

FIG. 8 shows a device similar to FIG. 7 during the elasticity measurement of tissue with laterally variable elasticity.

FIG. 9 shows a further embodiment of a device for elasticity measurement via a reaction force.

FIG. 10 shows a time-dependent pressure profile for generating a corresponding fluid jet.

FIG. 11 is a schematic view of a hand-guided surgical or diagnostic apparatus equipping a device in accordance with a development of the invention.

FIGS. 12 and 13 are schematic sectional views of measuring heads of surgical or diagnostic instruments, in which the device for elasticity measurement is combined in each case with an imaging optics.

FIG. 14 is a schematic sectional view of an endoscope which contains a device for elasticity measurement in accordance with a development of the invention.

FIG. 15 is a schematic illustration of a distal end of an endoscope having a rotatable probe tip, in which a device for elasticity measurement is integrated.

### DESCRIPTION OF THE PREFERRED EXEMPLARY EMBODIMENTS

For the improved understanding of the present invention, reference will be made hereinafter to the preferred exemplary embodiments illustrated in the drawings, these being described on the basis of specific terminology. However, it should be noted that the scope of protection of the invention is not to be limited thereby, since changes and further modifications to the presented devices and the described method as well as further applications of the invention as presented in the drawings are considered as would normally occur to the person skilled in the art now or in the future.

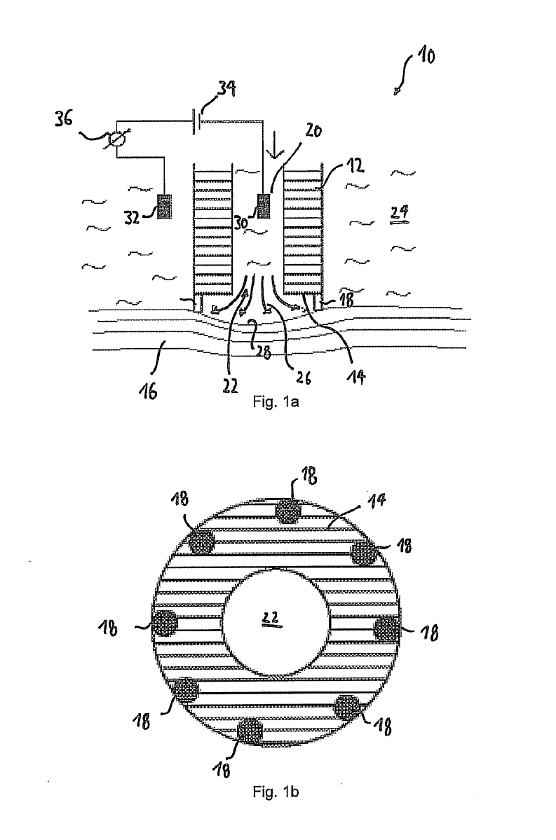

FIG. 1a shows a schematic sectional view of a device 10 for measuring the elasticity of tissue of a living human or animal, i.e. for in vivo elasticity measurement. FIG. 1b shows a view from below of the device 10. The shown device 10 and the deviating variants thereof described hereinafter can be combined with a diagnostic or surgical instrument that is to be guided by hand or by a surgery robot or can be formed as part thereof.

The device has a body 12, which is also referred to as a measuring head, on the lower end of which in the illustration of FIG. 1a there is formed an end face 14. The end face 14 is arranged facing towards a portion of the tissue 16 of which the elasticity is to be measured. Spacers 18 are provided on the end face 14 and, as can be seen particularly in FIG. 1b, are arranged annularly and together define a bearing surface for the device, by means of which the device can be placed against the tissue 16. In the embodiment the virtual ring along which the spacers 18 are arranged has a diameter d of 1 cm, such that the bearing surface occupies an area of approximately 0.8 cm². In the operating state shown in FIG. 1a the device 10 is placed against the tissue 16 such that all spacers 18 are in contact with the tissue. The spacers 18 thus constitute an exemplary embodiment of the means mentioned in the introduction for positioning the device 10 in relation to the macroscopic sample, i.e. the tissue 16.

In a middle portion of the device 10, a channel 20 is formed which ends in the end face 14 in an outlet 22. The outlet 22 is not specified in greater detail in the schematic illustration of FIG. 1a, and in specific embodiments may be a suitably designed nozzle or the like.

A fluid, in the shown exemplary embodiment a physiological saline solution 24, is pressed through the channel 20 at a predetermined pressure, such that a jet of fluid 26 exits from the outlet 22. As can be seen in FIG. 1a, this jet of fluid 26 is directed to the tissue 16 and leads to an elastic deformation thereof, wherein the deformed region is designated by the reference sign 28.

A first electrode 30 is arranged in the channel 20. In a radially outer region of the device 10 there is arranged a second electrode 32. The device comprises a voltage source 34, which applies a voltage between the first and the second electrode 30, 32. Lastly, a current measuring mechanism 36 is provided, with which the strength of a current flowing between the first and the second electrode 30, 32 can be measured.

The operating principle of the device from FIGS. 1a and 1b will be explained hereinafter. When a voltage is applied between the first and second electrode 30, 32, an ion current flows between these electrodes 30, 32 through the saline solution 24. At a given voltage the current strength is dependent on the size of the gap between the end face 14 of the device 10 and the surface of the tissue 16. This can be understood as follows. If the device 10 is located at a great distance from the tissue 16, the current strength is then only limited by the intrinsic resistance of the electrolyte, i.e. the saline solution 24. However, if the device 10 is moved increasingly towards the tissue 16, current is increasingly cut off by narrowing the gap between the end face 14 and the surface of the tissue 16, which leads to a drop of the current strength measured by the current mechanism 36.

This principle is known per se for the purposes of a distance measurement from scanning ion conductance microscopy (SICM). In the case of SICM fine pipettes are guided typically over microscopic samples. With the aid of a control circuit the pipette can be moved here in the vertical direction such that a constant current flows, which already represents a certain degree of the cutting off. The vertical movement performed by the pipette during this process is recorded and represents the height profile of the sample. Surface reliefs can thus be detected contactlessly. SICM technology is described for example in Hansma, P. K; Brake, B; Marti, O; Gould, S. A.; Prater, C. B. The scanning ionconductance microscope. Science 1989, 243(4891), 641-643 and Korchev, Y E., C. L. Bashford, M Milovanovic, L Vodyanoy, and M. J. Lab, Scanning ion conductance microscopy of living cells. Biophysical Journal, 1997. 73(2): p. 653-8.

SICM technology is used generally for microscopic samples in the laboratory, in which cell samples are typically moved in a petri dish relative to the pipette on an X-Y scanning table and the surface is scanned. In Sanchez D., N Johnson, C. Li, P. Novak, J. Rheinlaender, Y. Zhang, et al, Noncontact measurement of the local mechanical properties of living cells using pressure applied via a pipette. Biophysical Journal, 2008. 95(6): p. 3017-27 the mechanical properties of a microscopic sample, specifically of a cell membrane, were also examined by exerting a hydrostatic pressure on the microscopic sample by the pipette.

In the embodiment of FIG. 1a the spacers 18 are selected such that the gap between the end face 14 and the surface of the tissue 16 is already so small that there is a significant cutting off of the ion current. If a pressure is now applied in the channel 20 and a jet of fluid 26 is generated, the surface of the tissue 16 is locally deformed, as shown by the reference sign 28, such that the gap between the end face and the surface of the tissue 16 is increased. This leads to a rise of the ion current detected by the current measuring mechanism 36. In this respect the ion current is a variable that is characteristic of the extent of the deformation 28 of the tissue 16.

With predefined pressure in the channel 20 and with a predefined distance between the outlet or the nozzle 22 and the tissue 16, the extent of the deformation 28 is in turn a measure for the elasticity of the tissue 16, in particular the E-modulus thereof. In this respect the elasticity of the tissue 16 at the examined position can be concluded very precisely by the current strength measured using the current measuring mechanism 36. The exact relationship between the measured current and the elasticity can be determined by comparison or calibration measurements. Tests performed by the inventors have shown that the ion current measurement is a very sensitive measure for the elasticity, which in particular has the potential to differentiate between healthy tissue and tumour tissue.

For the reproducibility of the measurements, it is of particular importance for the distance between the end face 14 and the tissue 16, or between the outlet 22 and the tissue 16 to be predefined sufficiently precisely by the spacers 18 without impairing the handling of the device 10, which is typically hand-guided (as explained later). The device 10 actually needs only to be simply placed on the tissue 16 via the spacers 18, whereby the predetermined distance between the outlet 22 and the end face 14 on the one hand and the tissue 16 on the other hand is produced automatically.

The structure shown merely schematically in FIGS. 1a and 1b is to be understood only by way of example and is in no way limiting. Further preferred variants will be described hereinafter, likewise merely schematically.

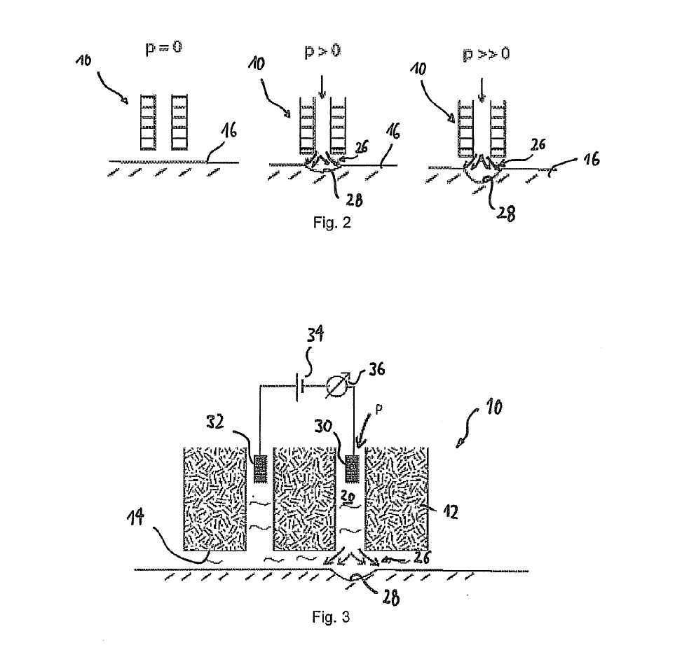

FIG. 2 schematically shows how the extent of the deformation 28 changes depending on the pressure within the fluid channel 12. To the far left in FIG. 2 the situation is shown in which no artificial pressure is produced in the channel 20 and the tissue accordingly is not artificially deformed. The middle image shows a situation with an artificial pressure of moderate level, which leads to a comparatively small deformation 28 of the tissue 16. To the far right a situation is schematically shown in which an even greater artificial pressure is produced in the channel 20 and leads to a comparatively large deformation 28. In an advantageous embodiment the pressure in the channel 20 can be controlled arbitrarily in order to generate suitable jets of fluid. By way of example, it is possible with comparatively rigid samples, i.e. with samples having a comparatively high E-modulus, to work with a higher pressure than with softer samples in order to determine the specific elasticity value with good accuracy. It is also advantageous when a mechanism for controlling the pressure is provided, with which a time-dependent pressure profile can be provided and the ion current likewise is measured in a time-resolved manner. Viscoelastic properties can thus also be examined.

FIG. 3 shows an embodiment that is similar to that from FIG. 1a, but in which the second electrode 32 is arranged within an indentation or a channel 38 leading into the end face 14. This indentation or channel 38 can also be referred to as a “passive opening” because no artificial pressure or negative pressure is generated therein. The arrangement of the second electrode 32 in the passive opening 28 in the end face 14 is of particular advantage insofar as the second electrode 32 itself does not protrude into the jet, but can be arranged in an optimal spatial position in relation to the first electrode 30 in order to provide a good measurement sensitivity. It can be seen that even small deformations 38 in the tissue that would be more difficult to detect with a second electrode 30 arranged outside the device 10 similarly to the manner shown in FIG. 1a can be determined in the tissue.

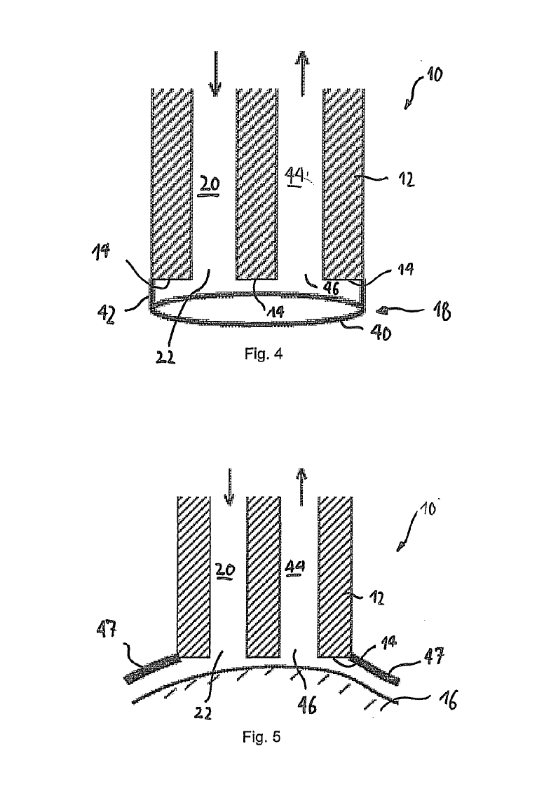

In FIGS. 4 and 5 alternative embodiments for spacers 18 are shown. In the embodiment of FIG. 4 the spacer is formed by a wire ring 40, which is distanced from the end face 14 of the device 10 via retaining elements 42. The operating principle of the wire ring 40 is similar to that of the spacers 18 shown in FIGS. 1a and 1b, which are arranged in an annular pattern. The embodiment of FIG. 4, besides a fluid channel 20, in which a positive pressure prevails, also shows a channel 44, which has an inlet 46 in the end face 14 and in which a negative pressure prevails. The electrodes (not shown in FIG. 4) may then be arranged in the channels 20, 44. The strength of the ion current is in turn characteristic of the deformation of the tissue (not shown in FIG. 4) caused by the jet of fluid exiting from the outlet 22 of the channel 20.

FIG. 5 shows a similar embodiment, but in which instead of the wire ring a flexible rubber lip 47 is provided. The rubber lip 47 is particularly flexible at its radially outer portions and adapts to the tissue 16, and at the same time stiffer radially inner portions ensure that the device 10 as a whole is held at a predetermined distance from the tissue 16. In the embodiment of FIG. 5 as well, the fluid, for example a physiological saline solution, is sprayed through a channel 20 and an outlet or a nozzle 22 onto the tissue 16 and is aspirated by a negative pressure channel 44. The rubber lip 47 prevents considerable quantities of the fluid from escaping into the surrounding environment.

It is also possible for the rubber lip 47 to bear so closely against the sample 16 that no fluid can escape between the rubber lip 47 and the sample 16. In this case the jet of fluid is dammed up and the fluid flow comes to a complete standstill. This is an example of the interaction, mentioned in the introduction, between the jet of fluid and the sample, on account of which the sample is deformed and in this case the jet of fluid is dammed up. More specifically, a fluid bubble enclosed by the sample is formed, the size of said bubble being dependent on the elasticity properties of the sample. The situation of a dammed-up fluid may also easily occur as a result of the fact that the end face rests on the sample in an annular region around the outlet and the fluid is thus enclosed. With a suitable arrangement of the electrodes, the extent of the deformation can be determined in this case as well by an ion current measurement.

It should be taken into consideration that the term “fluid bubble enclosed by the sample” is not intended to suggest that the fluid is enclosed by the sample alone, but instead the “fluid bubble” is generally delimited on one side by a portion of the device 10 and on the other side by the deformed sample 16. A fluid bubble of this type assimilates the deformation area 28 from FIG. 1a, except for the fact that the end face 14 by way of example would rest directly on the tissue 16 in an annular area around the outlet 22, such that the fluid would be enclosed in the deformation area.

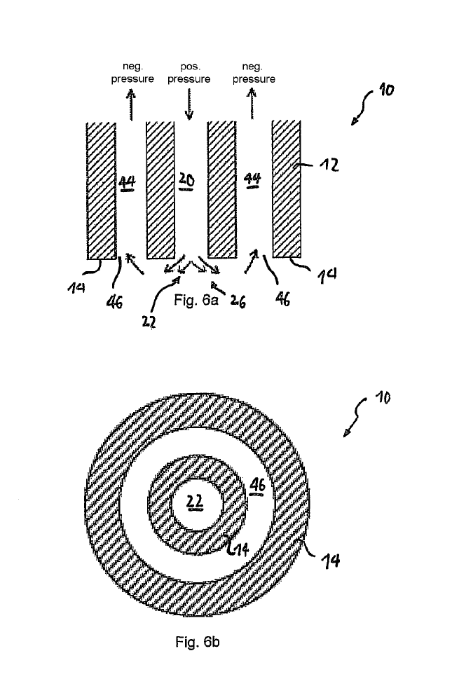

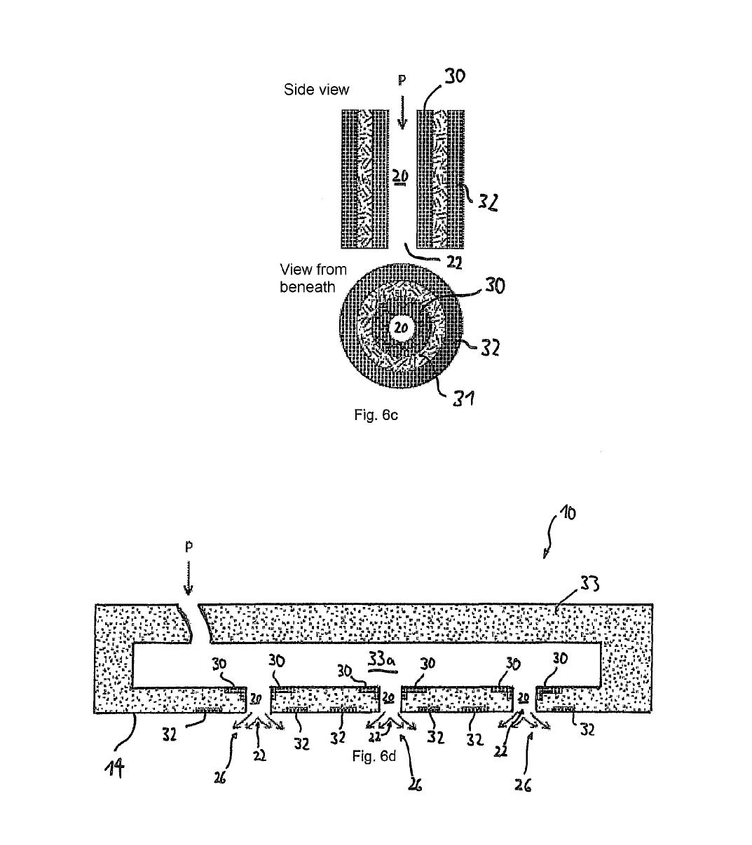

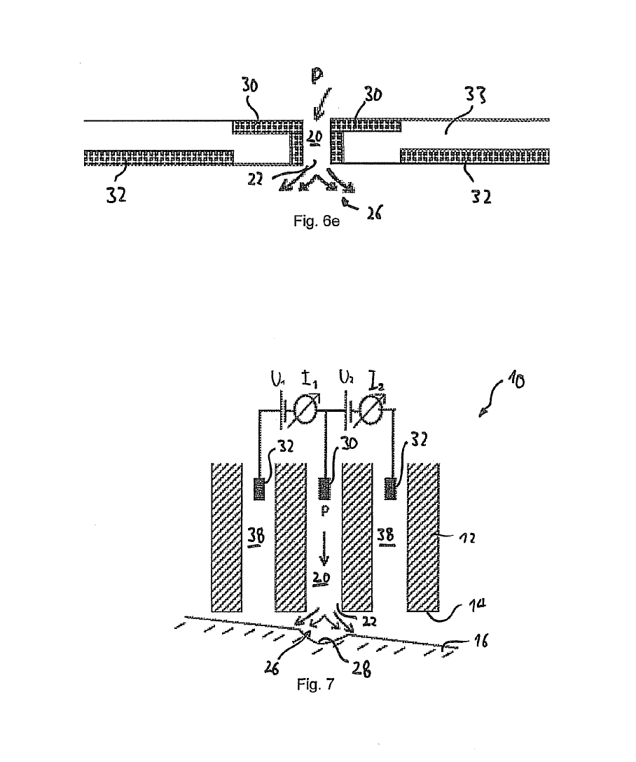

FIGS. 6a and 6b show, respectively, a further embodiment in cross section and in a view from below, in which a central channel 20 is provided with overpressure and a radially outer annular channel 44 is provided with negative pressure. This structure leads to a radially outwardly directed fluid flow 26 from the central channel 20 through the gap between the end face 14 of the device 10 and the tissue (not shown in FIGS. 6a and 6b) into the annular channel 44.

In FIGS. 1a and 3, the first and the second electrode 30, 32 are illustrated merely schematically in order to explain the operating principle. As is shown in FIG. 6c, the first electrode 30 may be formed for example by a conductive coating of the fluid channel 20. The second electrode 32 may also be formed by a conductive coating of a portion of the device 10, a coating of the outer peripheral surface in the shown exemplary embodiment. An insulator 31 is arranged between the first and the second electrode 30, 32.

In an advantageous development the device comprises a plurality of outlets 22, which are arranged side by side, as is shown in FIG. 6d. In FIG. 6d the device 10 has an insulating housing 33, on the underside of which an end face 14 is formed. A plurality of outlets 22 are arranged in the end face 14. Although merely three outlets can be seen in the sectional illustration of FIG. 6d, more than 10, in particular more than 50, and even more than 100 outlets 22 of this type can readily be arranged side by side in the end face. The outlets 22 are connected via channels 20 to a cavity 33a within the housing, which is acted on by a pressure during operation of the device 10 in order to generate fluid flows 26, which exit from the outlets 22. A first electrode 30 is arranged in the region of each channel 20. A second electrode 32 is also provided in the end face 14 for each outlet 22 and surrounds the respective outlet 22 annularly. For the function however, it is not absolutely necessary for the second electrode 32 to completely or annularly surround the outlet 22. Although this is not shown in FIG. 6d, a current measuring mechanism is provided, which can determine the current flow through each of the individual second electrodes 32, such that a respective ion current measurement can be taken at each outlet 22.

The device 10 from FIG. 6d can be produced easily and economically by means of established microfabrication processes, i.e. with use of processing steps such as etching, deposition of metal layers, polishing and the like. In particular, the portion of the housing 33 that forms the end face 14 having the outlets 22 may consist of silicon, for which there are extraordinarily well established processing possibilities.

In the embodiment of FIG. 6d the end face 14 is placed directly against the tissue (not shown). Since in this state the second electrode 32 is covered by the tissue (not shown), it is not possible for a significant ion current to flow between the first and the second electrode 30, 32. As soon as an overpressure is formed in the cavity 33a, fluid flows 26 exit from the outlets 22 and locally deform the issue (not shown in FIG. 6d). As a result of this deformation, the tissue can be lifted from the end face 14, and the second electrode 32 can be partially exposed, such that an ion current can flow between the first and the second electrodes 30, 32. The greater the extent of the deformation, the greater is the portion exposed of the respective second electrode 32, and the greater is the measured ion current. A particular feature in this embodiment lies in the fact that the deformation can be measured simultaneously in a spatially resolved manner at a multiplicity of points, corresponding to the positions of the outlets 22.

FIG. 6e shows a slightly enlarged illustration of a channel 20 having an associated outlet 22 and associated first and second electrode 30, 32. In the case of FIG. 6e, the first electrode, by contrast with the embodiment of FIG. 6d, extends along the entire length of the respective channel 20. For the spatially resolved measurement, it is important for an associated second electrode 32 to be provided actually for each outlet 22, but at least for the majority of the outlets 22, such that the deformation of the tissue can be measured in the region of this outlet 22. However, it is not necessary for each outlet 22 to be assigned a dedicated first electrode. Instead, it would also be possible by way of example to provide only a sole first electrode 30 in the cavity 33a.

FIG. 7 shows a schematic sectional view of a further embodiment of the device 10. The embodiment of FIG. 7 again has a central channel 20, in which the first electrode 30 is arranged and in which a positive pressure is produced. This embodiment also comprises two second electrodes 32, which are arranged in different, radially outer portions of the end face 14, more specifically in corresponding indentations 38 in the end face 14, which are located in a radially outer portion of the device 10. The currents I₁ and I₂ through each of the two second electrodes 32 are measured separately. As in the previously described embodiments, the current I₁ and the current I₂ at a predefined distance between the end face 14 and the tissue 16 are a measure of the extent of the deformation 28. The current values I₁ and I₂, however, may additionally be used when positioning the device 10 in relation to the tissue 16. By way of example, FIG. 7 shows a situation in which the end face 14 is tilted relative to the surface of the tissue 16, such that the gap located therebetween has an inhomogeneous width. This could be detected in the situation of FIG. 7 on the basis of the fact that the current I₂ is greater than the current I₁. This measurement could also be taken before a positive pressure is actually produced in the central channel 20 with corresponding deformation, such that the currents I₁ and I₂ can be attributed to the positioning (and not to the deformation). As is clear from this exemplary embodiment, the at least one first electrode 30 and the at least one second electrode 32 can be used not only to measure the extent of the deformation 28, but also to position the device 10 in relation to the tissue 16. In particular, the distance of the device 10 from the tissue 16 can be determined by the currents I₁ and I₂, even without deformation of the tissue 16, because, as the device 10 is moved towards the tissue 16, a cut-off effect of the ion current occurs and can be detected. In this respect, the same electrodes that are used to measure the deformation of the sample can also be used to position the device 10 in relation to the tissue 16 by measuring the distance of the end faces 14 from the tissue 16 and therefore also the distance of the outlet 22 of the central channel 20 from the tissue 16. It should be taken into consideration that the radially outer indentations 38 in FIG. 7 are separate indentations, for example are not part of an annular indentation.

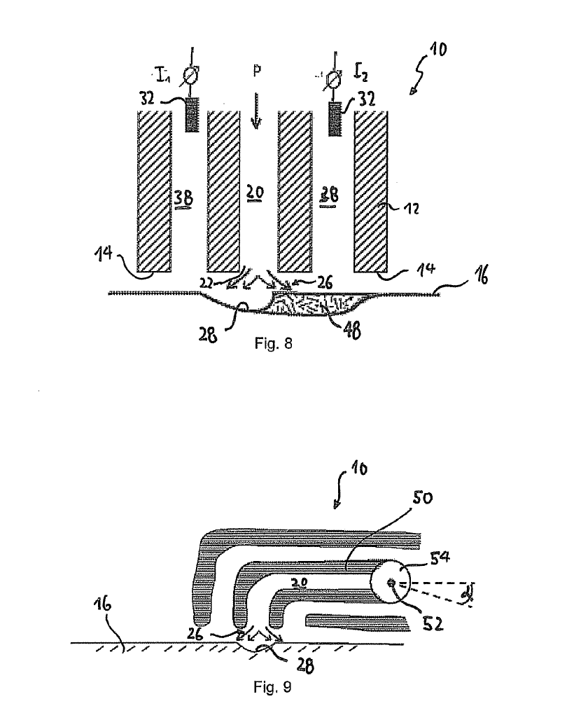

In FIG. 8 the same structure as in FIG. 7 is shown, but in another application. In the case of FIG. 8 it is assumed that the device 10 is arranged correctly in relation to the tissue 16, i.e. in such a way that the end face 14 of the device 10 is parallel to the surface of the tissue 16. This can be verified for example on the basis of the fact that the currents I₁ and I₂ are identical without positive pressure in the central channel 20. However, with generation of a pressure in the central channel 20 and with generation of an associated jet of fluid 26, this may then cause the currents I₁ and I₂ to no longer be identical, and in the shown exemplary embodiment may cause the current I₂ to be smaller than I₁. This difference in the currents I₁ and I₂ is to be attributed to an inhomogeneous deformation 28, which is in turn caused by an inhomogeneous local elasticity distribution of the tissue 16, for example by a local stiffer region in the tissue 16, which is characterised in FIG. 8 by reference sign 48.

It is clear from the description of FIG. 7 and FIG. 8 that the use of at least two second electrodes assigned to the same outlet 22 increases the functionality of the device 10 from many viewpoints, and in particular not only allows the measurement of a local elasticity, but also of a lateral variation of the elasticity. The greater the number of second electrodes that are used, the greater is the lateral resolution of the measurement in this respect. Embodiments having two to six, particularly preferably those having three to five second electrodes per outlet 22 are currently particularly preferred. Irrespectively of this, as shown above with reference to FIG. 6, a spatially resolved measurement can additionally be achieved by a plurality of outlets or inlets.

Although the ion current measurement is the currently preferred variant for measuring the extent of the deformation 28, the invention is not limited thereto. By way of example, it is also possible, with the aid of a flowmeter, to measure the volumetric flow of the fluid with a predefined distance between the device 10 and the tissue 16 and at a predefined pressure. The softer the tissue, i.e. the stronger the deformation, the greater is the gap between the device 10 and the tissue 16 and the lower is the flow resistance, which in turn leads to an increased volumetric flow. Alternatively, the pressure can also be measured with predefined flow rate or volumetric flow, or pairs of pressure-volumetric flow values can be determined. In all of these cases the relationship between pressure and volumetric flow is characteristic of the elasticity of the tissue.

It is also possible to directly measure the force exerted via the jet of water onto the tissue, as illustrated schematically in FIG. 9. In the embodiment of FIG. 9 the channel 20 with the pressurised fluid is located in a spring-mounted lever 50, which is pivotably mounted about an axis 52 and can be pivoted against a restoring force of a torsion spring 54. The restoring force of the jet of fluid 26 leads to a torque on the lever 50 and a corresponding pivoting about an angle α, at which the restoring force of the torsion spring 54 and the torque are balanced on account of the reaction force. When the tissue 16 deforms as a result of the jet of fluid 26 and evades said jet of fluid, the reaction force and therefore the angle α reduces. In this respect the angle α is also a measure for the extent of the deformation 28 of the tissue 16.

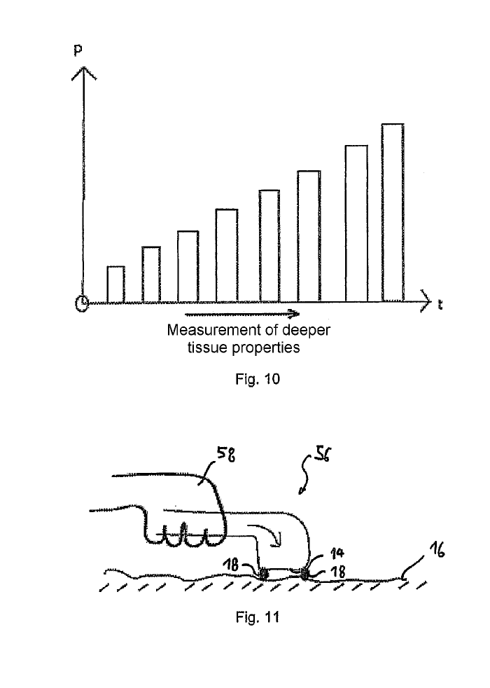

As already mentioned above, it is advantageous in many embodiments when the pressure of the fluid can be freely predefined or controlled. In particular with a freely predefinable temporal pressure profile, more complex parameters of the tissue properties can be determined, wherein suitable models of the continuum mechanics can be applied. A comparatively simple pressure profile is shown in FIG. 10, in accordance with which the pressure is pulsed rectangularly and is raised in a ramp-like manner. As a result of this increasing pressure it is made possible to reach the deeper regions of the tissue successively and thus obtain three-dimensional information with regard to the tissue rigidity.

The embodiments of the device 10 for measuring the elasticity of macroscopic samples, in particular human tissue 16, discussed in the above figures can be economically provided and are easily integrated into diagnostic or surgical instruments that are to be guided by hand, as is shown by way of example in FIG. 11. In FIG. 11a a hand-guided instrument 56 is shown, which contains a device 10 in accordance with one of the above-described embodiments, of which only the end face 14 and the spacers 18 can be seen in FIG. 11. This instrument 56 can be guided by hand (reference sign 58) over the tissue 16 by the doctor in order to measure elasticity properties in a spatially resolved manner. A particular advantage of the device 10 of the invention lies in the fact that the structure is very simple, i.e. the equipment outlay is limited substantially to channels, means for pressure generation, electrodes, a voltage source and a current-measuring apparatus. These components not only can be produced easily and economically, but on account of their low spatial requirement also allow simple integration into hand-guided apparatuses having further diagnostic or surgical modes.

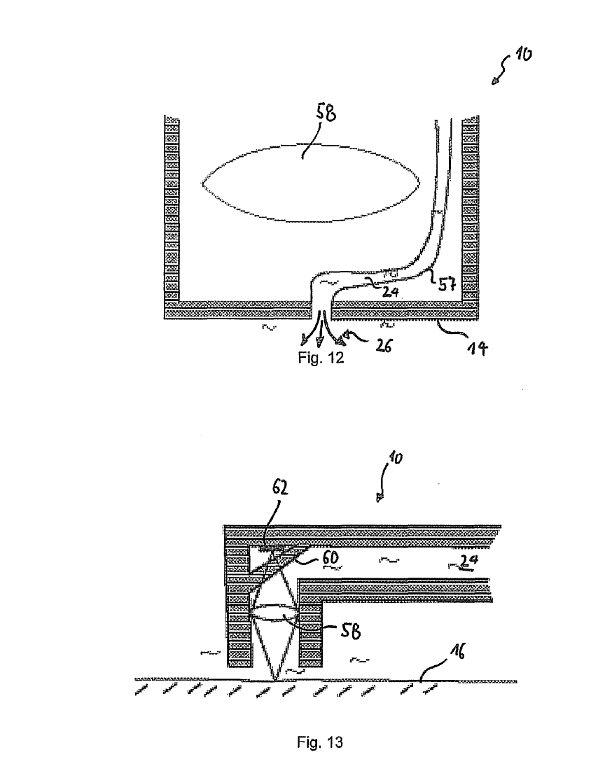

By way of example, FIG. 12 shows the measuring head of an apparatus similar to the apparatus 56 of FIG. 11, in which the channel 20 is formed by a liquid-filled tube 57. This tube makes it possible for example to guide the fluid around an imaging optics, for example a lens 58, such that the device 10 can be combined comparatively easily with an imaging optics. By way of example, it is possible simultaneously to record optical images of the examined tissue 16 and to record elasticity values locally. With a suitable data processing unit (not shown), the optical images and the elasticity values can then be coregistered or superimposed accordingly. By way of example, an optical image can be output, in which areas having special elasticity values, elasticity fluctuations, etc. are highlighted in order to facilitate the diagnosis of tumours.

A further exemplary embodiment, in which the device 10 is combined with an imaging optics, is shown in FIG. 13, in which a lens 58, a window 60, and an optical sensor 62 likewise are provided in the apparatus 56. In the embodiment of FIG. 13, part of the light path thus passes through the fluid 24, which is used to artificially deform the tissue 16.

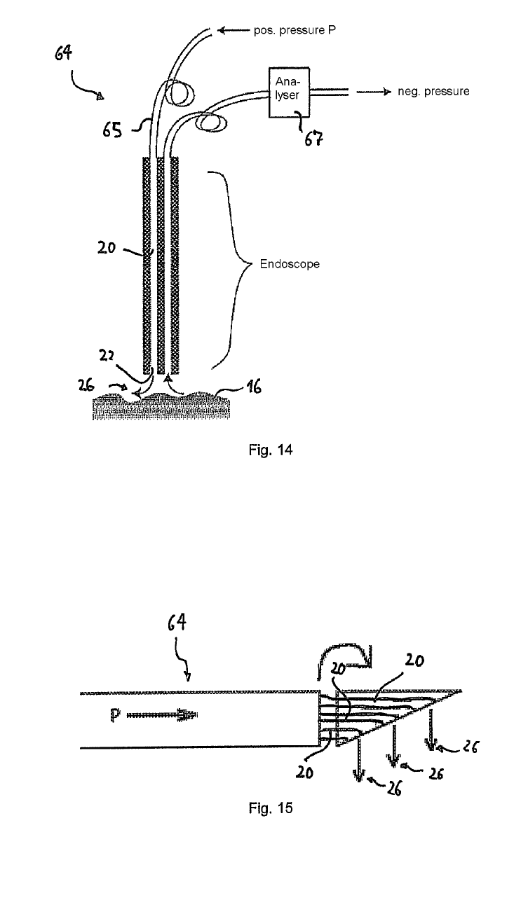

The comparatively low equipment outlay makes the device 10 for elasticity measurement also ideally suited for endoscopic or laparoscopic applications. FIGS. 14 and 15 schematically show exemplary embodiments. FIG. 14 shows an endoscope 64 having a channel 20, from the outlet 22 of which a jet of fluid 26 is discharged in order to locally deform the tissue 16. The positive pressure required for this purpose can be generated outside the endoscope 64 and supplied via a tube 65. Although this is not shown in FIG. 14, the endoscope, besides a camera, also has further functions, for example for the resection or ablation of tissue by means of a probe, tweezers, a cutting-edge, tongs, or the like. These tools may also be supplied via an HF terminal with an electrical HF signal in order to additionally obtain electrical-surgical functions. Here, endoscopic or laparoscopic apparatuses that are designed for water jet surgery, i.e. are capable of cutting tissue using a fine jet of water, are particularly preferred. In this case, the components provided anyhow for water jet surgery can also be used for pressure generation, fluid supply, etc. for the device 10 for elasticity measurement.

FIG. 14 also shows a channel 44 having a negative pressure, by means of which ablated tissue can be aspirated and supplied to an analyser 66. This channel 44 therefore also can be used not only for the elasticity measurement, but also for the recovery of tissue samples.

Lastly, FIG. 15 shows a schematic view of a distal end of an endoscope 64, in which the device 10 is formed in the manner of a rotatable probe tip 68. In FIG. 15 merely the channels 20 and the associated jets of fluid 26 are schematically illustrated.

The described device is able not only to measure the E-modulus or a viscosity, but can also be used to determine complex mechanical behaviour patterns. Examples of this include the profile over time or the frequency dependency of the tissue response to a mechanical stimulation, or the occurrence of certain patterns in this time or frequency profile. These behaviour patterns in a certain way form a “mechanical fingerprint” of the tissue. A behaviour pattern of this type can be used to identify or differentiate certain tissue types.

As mentioned in the introduction, the described device allows the surgical or diagnostic element to be used for differentiation of tissue, in particular for the identification of intraoperative tumours, nerves, blood vessels, etc.

Although preferred exemplary embodiments have been presented and described in detail in the drawings and in the above description, these should be considered as purely exemplary and not limiting the invention. It should be noted that only the preferred exemplary embodiments are illustrated and described, and all changes and modifications lying currently and in the future within the scope of protection of the claims are protected.

### LIST OF REFERENCE SIGNS

10 device for measuring the elasticity of a sample
12 body of the device 10
14 end face
16 tissue
18 spacer
20 fluid channel
22 outlet
24 electrolyte
26 stream of fluid
28 deformation
30 first electrode
31 isolator
32 second electrode
33 housing
33a cavity in housing
34 voltage source
36 current measuring mechanism
38 channel (passive opening)
40 wire ring
42 retaining element
44 negative pressure channel
46 inlet
47 flexible rubber lip
48 locally rigid area in tissue 16
50 lever
52 pivot axis of the lever 50
54 torsion spring
56 hand-guided diagnostic or surgical instrument
57 tube
58 lens
60 window
62 optical sensor
65 tube
67 analyser

1. A diagnostic or surgical instrument for guiding by hand or by a surgery robot, having a device for measuring the elasticity of tissue of a living human or animal, wherein the device comprises the following:
at least one outlet for a jet of fluid and/or one inlet for aspiration of a stream of fluid, means for positioning the device in relation to the tissue in such a way that
the outlet and/or the inlet and/or
an end face of the device
is/are at a predetermined distance from the tissue or is/are at a distance therefrom that can be determined by said means, and
a mechanism for measuring a variable that is characteristic of the extent of a deformation of the tissue on account of an interaction of the tissue with the jet of fluid and/or the aspirated stream of fluid, wherein this variable is determined
by an ion current in the deformation area of the tissue, or
the volumetric flow of the fluid itself or—if the fluid is enclosed by the elastic sample—by a change in volume and associated change in pressure in the enclosed fluid.

2. The instrument according to claim 1, which is also designed for water jet surgery.

3. The instrument according to claim 1, which also comprises one or more of a probe, tweezers, a cutting edge and/or tongs, in particular in each case having an HF terminal.

4. The instrument according to claim 1, further comprising a camera, wherein the instrument is connected or can be connected to a data processing unit, which relates the elasticity values measured in a location-dependent manner to the images recorded by the camera.

5. The instrument according to claim 1, wherein the instrument is an endoscopic or laparoscopic instrument.

6. The instrument according to claim 5, in which the device for elasticity measurement is formed by a rotatable measuring head of the endoscopic or laparoscopic instrument.

7. The instrument according to claim 1, wherein the end face can be arranged parallel or approximately parallel to the surface of the tissue so as to form a gap with the surface of the tissue or rest against the surface of the tissue.

8. The instrument according to claim 1, having a continuous or interrupted bearing surface, by means of which the device can be placed on the tissue during the measurement of the elasticity, wherein the bearing surface occupies an area of at least 10 mm².

9. The instrument according to claim 1, in which the fluid is an electrolyte, in particular a saline solution.

10. The instrument according to claim 7, in which the outlet and/or the inlet is or are arranged in the end face.

11. The instrument according to claim 1, wherein
the device
comprises at least one first and at least one second electrode, between which a voltage can be applied, and
comprises a current measuring mechanism in order to measure an electric current flowing between the first and the second electrode,
wherein the at least one first electrode and the at least one second electrode are arranged such that, with suitable positioning of the device in relation to the tissue, at least some of the electric current can be formed by an ion current in an electrolyte in a gap between the device and the tissue.

12. The instrument according to claim 11, in which the at least one first electrode is arranged in a fluid channel that is connected to the outlet, and in which the at least one second electrode is arranged in a fluid channel that is connected to the inlet, wherein the at least one first electrode and/or the at least one second electrode is/are preferably formed by a conductive coating of at least part of the respective fluid channel.

13. (canceled)

14. The instrument according to claim 11, which comprises at least two second electrodes, preferably at least three second electrodes and particularly preferably at least four second electrodes, wherein a voltage can be applied between each of the second electrodes and the at least one first electrode, wherein the at least one first electrode is arranged in a radially inner portion of the end face, and at least two second electrodes are arranged in or in the vicinity of different, radially outer portions of the end face, and in which the positioning means are designed to detect a tilting of the end face in relation to the surface of the tissue by comparison of the currents through the at least two second electrodes.

15. (canceled)

16. (canceled)

17. The instrument according to claim 11, wherein the at least one first and the at least one second electrode are arranged in relation to the outlet and/or the inlet such that at least some of the electric current between the at least one first and the at least one second electrode can be formed by an ion current in the deformed region of the tissue, and
wherein the variable characteristic of the deformation is formed by the current flow between the at least one first and the at least one second electrode.

18. The instrument according to claim 1, in which the means for positioning the device in relation to the tissue comprise at least one spacer.

19. (canceled)

20. (canceled)

21. The instrument according to claim 11, in which the second electrode is arranged on the end face, and
in which the means for positioning the device in relation to the tissue is formed preferably by the end face to be applied against the tissue.

22. (canceled)

23. The instrument according to claim 1, in which the outlet is formed by a nozzle of which the cross section, shape and/or exit angle is adjustable.

24. The instrument according to claim 1, having a mechanism for generating a pressure in a channel that is connected to the outlet and/or having a mechanism for generating a negative pressure in a channel that is connected to the inlet, in which the mechanism for generating the pressure is suitable for generating a time-dependent pressure profile and/or the mechanism for generating the negative pressure is suitable for generating a time-dependent negative pressure profile, wherein the mechanism for measuring the variable characteristic of the deformation of the tissue is suitable for measuring this variable in a time-resolved manner.

25. (canceled)

26. (canceled)

27. The instrument according to claim 11, which comprises at least two second electrodes and which comprises a data processing unit or is connected to a data processing unit suitable for determining a lateral variation in the elasticity by comparison of the currents through the at least two second electrodes.

28. (canceled)

29. A method for measuring the elasticity of tissue of a living human or animal with the aid of a device for measuring the elasticity, which device is part of a diagnostic or surgical instrument, said method comprising the following steps:
positioning the diagnostic or surgical instrument by hand or with the aid of a surgical robot in relation to the tissue,
in such a way that
an outlet and/or an inlet of the device and/or
an end face of the device is/are located at a predetermined distance from the tissue, or
with determination of the distance of the outlet and/or of the inlet from the tissue, generating a jet of fluid through the outlet in the direction of the tissue and/or aspiration of a stream of fluid through the inlet, and
measuring a variable characteristic of the deformation of the tissue on account of an interaction of the tissue with the jet of fluid and/or the aspirated stream of fluid,
wherein this variable is determined by
an ion current in the deformation area of the tissue, or
the volumetric flow of the fluid itself or—if the fluid is enclosed by the elastic tissue—by a change in volume and associated change in pressure in the enclosed fluid.

30.-32. (canceled)
